(19) United States
(12) Patent Application Publication
(10) Pub. No.: US 2008/0249054 A1
(43) Pub. Date: Oct. 9, 2008

(54) METHOD OF MEASURING LIPID DROPLETS AND APPLICATIONS OF USING THE SAME

(75) Inventors: Carole Sztalryd-Woodle, Bethesda, MD (US); Ming Bell, Laurel, DE (US); Hong Wang, Ellicott City, MD (US)

Correspondence Address: MERCHANT & GOULD PC, P.O. BOX 2903, MINNEAPOLIS, MN, 55402-0903, US

(73) Assignee: UNIVERSITY OF MARYLAND, BALTIMORE, Baltimore, MD (US)

(21) Appl. No.: 12/061,773

(22) Filed: Apr. 3, 2008

Related U.S. Application Data
(60) Provisional application No. 60/921,811, filed on Apr. 4, 2007.

(51) Int. Cl. A61K 31/70; C12Q 1/02; A61P 43/00; G01N 33/53

(52) U.S. Cl. 514/44; 435/29; 435/7.92

(57) ABSTRACT

The invention relates to methods of screening agents for the ability to regulate lipid metabolism and using said agents to treat diseases or disorders related to lipid metabolism (e.g., obesity, diabetes [non-insulin and insulin dependent], hypertension, coronary artery disease, hyperlipidemia (e.g., LDL, TAGs), hypolipidemia (e.g., HDL), lipid metabolism disorders, lipid deposition disorders, lipodystrophies). In specific embodiments, the invention relates to using a PAT family protein to screen agents for the ability to regulate lipid metabolism and using the same to treat diseases or disorders related to lipid metabolism. In further embodiments, the invention relates to high throughput screening (HTS) methods that can be used in the drug discovery process for screening agents for the ability to regulate lipid metabolism.

### STATEMENT REGARDING FEDERALLY SPONSORED RESEARCH OR DEVELOPMENT

This invention was made with Government support from funding from the Department of Veterans Affairs and sponsored research from the American Diabetes Association Career Development Award 1-05-CD-17. The Government has certain rights in the invention.

### TECHNICAL FIELD

The invention relates to compositions and methods of administering genetic modulation agents to cells, to methods of assaying lipid metabolism and to methods of determining biochemical pathway function that is useful to identify therapeutic interventions for treatment of metabolic diseases. The invention also relates to methods of screening for genes and agents useful for treating diseases or disorders related to metabolism, and in particular lipid metabolism associated with diabetes. The invention further relates to agents for treating diseases or disorders related to metabolism, and in particular lipid metabolism associated with diabetes.

### BACKGROUND OF INVENTION

### Diseases or Disorders Related to Lipid Metabolism

The striking surge in obesity has serious health consequences that increase risk for a number of diseases associated with increased morbidity and mortality, including, diabetes, insulin resistance, hypertension, and coronary heart disease (Meigs JB, 2002, Am J Manag Care 8:S283-S292). An important clue to the pathogenesis of these metabolic abnormalities is a consistent presence of ectopic fat, the accumulation of lipid droplets in non-adipose tissue (Eckel et al., 2005, Lancet 365(9468):1415-28). Normally, excess energy from dietary consumption in the form of non-esterified fatty acids (NEFA) is converted into triacylglycerol (TAG) and stored in lipid droplets within adipose tissue, a highly specialized and expandable organ. Poorly understood defects in fat storage lead to chronically elevated levels of circulating NEFA and result in accumulation of ectopic fat, most prominently in liver, muscle, and pancreas. There is a correlation of excess lipid droplets with disorders including insulin resistance in skeletal muscle (Shulman, G. I, 2000, J. Clin. Invest. 106, 171-176), dysregulated insulin secretion (Zhou et al, 1995, J. Clin. Endocrinol. Metab. 80, 1584-1590; Prentki et al., 1992. J. Biol. Chem. 267, 5802-5810; Shimabukuro et al., 1998. Proc. Natl. Acad. Sci. USA 95, 2498-2502), and heart failure (Chiu et al., J. Clin. Invest. 107:813-822; Zhou et al., 2000, Proc. Natl. Acad. Sci. USA 97: 1784-1789; Molavi et al., 2004, Curr Opin Cardiol: 488-93). On the other hand, despite a well-established positive correlation of ectopic fat with diseases or disorders related to lipid metabolism, studies of endurance trained athletes found that increased intramyocellular lipid (IMCL) is positively correlated with insulin sensitivity. The size and intracellular distribution of the lipid droplets in muscle from insulin-sensitive athletes differs from that in insulin resistant patients (Machann et al., 2004, Diabetes Obes Metab:239-48; Russell AP, 2004, Int J Obes Relat Metab Disord. December; 28 Suppl 4:S66-71). Thus, a major unanswered question is whether the development of metabolic disease is not simply due to the existence of ectopic fat but rather due to alterations in gene and protein function to manage lipid metabolism including excesses and supply in healthy versus diseased tissue, and if so how those alterations can be modulated so as to provide treatments for patients.

### Lipid Droplets

Fat storage in tissues can be visualized by microscopic observation as intracellular lipid droplets and their presence is a commonly used morphological feature of cells (Murphy et al., 1999, Trends Biochem Sci. (3):109-15). Lipid droplets were long thought of simply as a triacylglyeride (TAG) and cholesterol ester core surrounded by a phospholipid monolayer, with a spherical shape giving the least surface area for the lipid volume. This simplistic view of lipid droplets has been replaced by one where lipid droplets are now thought of as being actively involved in lipid metabolism. Recent findings show that lipid droplets are not static, but are rather a metabolically active cellular component that interacts with several other components of the cell.

Ectopic fat deposition, the accumulation of fats in lipid droplets in tissues other than adipose tissue, develops in obese patients and is now recognized as a strong prognostic factor for the development of diseases or disorders related to lipid metabolism. The molecular mechanisms regulating the formation and metabolism of lipid droplets in non-adipose tissues and their dysfunction in pathophysiological states are not well understood. Proteomic studies indicate that lipid droplets are surrounded by a protein coat that provides an interface for lipid metabolic processes, including, for example, transport, lipogenesis, and lipolysis (Brasaemle et al., 2004, J. Biol. Chem. 279, 46835-46842; Liu et al., 2004, J. Biol. Chem. 279, 3787-3792; Wu et al., 2000, Electrophoresis 16, 3470-3482; Fujimoto et al., 2004, Biochim. Biophys. Acta 1644, 47-59; Ozeki et al., 2005, J. Cell Sci. 118, 2601-2611). Even more importantly, these studies identify a proteome “signature” for lipid droplets that consistently includes at least one member of the PAT protein family (originally named for perilipin, ADFP [adipose differentiation-related protein] and Tip47 [tail interacting protein of 47 kDa]). A PAT protein is always present and generally represents the most abundant lipid droplet protein (Sztalryd et al., 2006, J. Biol. Chem. 281, 34341-34348).

### PAT Protein Family

The mammalian PAT family includes five members: perilipin, ADFP, Tip47, S3-12, and PAT1 (Lu et al., 2001, Mamm. Genome 9, 741-749). The PAT proteins are defined by primary sequence homology and are well conserved within the family and across species (Miura et al., 2002, J. Biol. Chem. 277, 32253-32257). Recent proteomic studies revealed heterogeneity and tissue-specific differences in droplet-associated proteins (Brasaemle et al., 2004, J. Biol. Chem. 279, 46835-46842; Liu et al., 2004, J. Biol. Chem. 279, 3787-3792; Wu et al., 2000, Electrophoresis 16, 3470-3482; Fujimoto et al., 2004, Biochim. Biophys. Acta 1644, 47-59; Ozeki et al., 2005, J. Cell Sci. 118, 2601-2611). PAT protein distribution is clearly tissue-dependent. Perilipin and S3-12 are confined to adipose and steroidogenic tissues, while ADFP and Tip47 are ubiquitously distributed (Blanchette-Mackie et al., 1995, J. Lipid Res. 6, 1211-1226; Servetnick et al., 1995, J. Biol. Chem. 270, 16970-16973; Brasaemle et al., 1997, J. Lipid Res. 11, 2249-2263; Wolins et al., 2001, J. Biol. Chem. 276, 5101-5108). To date, a functional role regulating lipolysis has been shown for perilipin, the principal lipid droplet protein in adipose cells, mainly from studies of the perilipin null mouse that exhibits a lean phenotype (Martinez-Botas et al., 2000, Nat. Genet. 4, 474-479; Tansey et al., 2001, Proc. Natl. Acad. Sci. U.S.A. 98, 6494-6499). However, little is known about the function of the other PAT proteins, including the proteins associated with lipid droplets in non-adipogenic tissues, such as ADFP and Tip47. At least one property of both ADFP and Tip47 is TAG hydrolysis. Therefore, systems are needed that can enable studies where simultaneous down-regulation of multiple genes, and in particular PAT proteins, is obtained, such as ADFP and Tip47, using siRNA and the impact on cell metabolism including TAG hydrolysis can be characterized.

The lack of methods of administering gene modulation agents and of quantifying lipid droplets as well as lipid droplet protein properties to screen agents for the ability to regulate lipid metabolism and using said agents to treat diseases or disorders related to lipid metabolism represents a long felt need in the art that has not been met. The invention described herein represents an invention that addresses a long felt need, for methods and compositions useful to monitor and treat diseases or disorders related to lipid metabolism.

### BRIEF SUMMARY OF INVENTION

The invention relates to compositions and methods to administer agents to cells to modulate gene and/or protein activity associated with metabolism, including formation of ectopic fat, and to methods to characterize the resulting alteration in cellular metabolism including intracellular lipid droplets as well as insulin signaling. The invention also relates to therapeutic targets as well as therapeutic agents that alter or regulate cellular metabolism including lipid metabolism, and pharmaceutical compositions comprising the same, used to treat diseases or disorders related to metabolism including lipid and insulin metabolism. The invention further relates to methods of screening, including high throughput screening (HTS), agents for the ability to regulate metabolism including lipid and insulin metabolism.

In certain embodiments, the invention relates to compositions and methods to modulate gene and protein activity coupled to characterization of lipid droplets in a cell. In specific embodiments, the invention is drawn to modulating the expression level of one or more lipid associated proteins and characterizing the changes in cellular metabolism upon said modulation of expression, including quantification of lipid droplet characteristics in a cell.

In further embodiments, the invention relates to methods of screening agents for the ability to alter or regulate metabolism in a cell, including lipid and insulin metabolism. In specific embodiments, the invention is drawn to contacting an agent with a cell and modulating the expression level of one or more cellular proteins, including PAT family proteins, in said cell. In further specific embodiments, the invention is drawn to a method of altering the expression level of one or more PAT family proteins in a treated cell compared to the expression level of the same PAT family proteins in an untreated cell, wherein a difference between said expression levels is observed for the ability to alter or regulate cellular metabolism, including lipid and insulin metabolism. In yet further specific embodiments, the invention is drawn to compositions and methods of screening agents for the ability to regulate metabolism in a cell, including lipid and insulin metabolism, wherein said agent is an RNA interference agent including small inhibitory RNA (siRNA) and short hairpin RNA (shRNA) expression.

In further embodiments, the invention relates to agents identified by compositions and methods described herein.

In yet further embodiments, the invention relates to compositions and methods for treating a metabolic disease or disorder, including those related to lipid metabolism and insulin metabolism, comprising administering to a patient in need thereof one or more of the agents identified by the methods described herein. In specific embodiments, the invention is drawn to methods of treating a disease or disorder related to lipid and insulin metabolism including but not limited to the group consisting of obesity, diabetes [non-insulin and insulin dependent], hypertension, coronary artery disease, hyperlipidemia (e.g., LDL, TAGs), hypolipidemia (e.g., HDL), lipid metabolism disorders, lipid deposition disorders, and lipodystrophies.

In further embodiments, the invention is drawn to compositions and methods for quantifying lipid droplets in a mammalian cell. In specific embodiments, said mammalian cell may be, for example, a murine cell, a human cell or a nonhuman primate cell.

In particular embodiments, the invention is drawn to compositions and methods for quantifying lipid droplets in a mammalian cell comprising treating the cell and measuring the characteristics of the lipid compartment within said cell including measures of levels of BODIPY lipid staining in said cell.

In yet further embodiments, the invention is drawn to compositions and methods for quantifying lipid droplets in a mammalian cell comprising modulating the expression level of one or more PAT family proteins, wherein said PAT family proteins are selected from the group consisting of perilipin, adipose differentiation-related protein (ADFP), tail interacting protein of 47 kDa (Tip47), S3-12, and PAT1, and subjecting said mammalian cell to assay methods to characterize the lipid compartment of said mammalian cell. In specific embodiments, said PAT family proteins are ADFP and/or Tip47. In further specific embodiments, said PAT family protein may further comprise a label. In yet further specific embodiments, said PAT family protein may further comprise a second polypeptide. In yet even further specific embodiments, said second polypeptide is an enzyme or a fluorescent protein.

In further specific embodiments, the invention is drawn to compositions and methods comprising detecting one or more PAT family proteins, wherein said detection of a PAT family protein is achieved by fluorescence, luminescence, ELISA, radioimmunoassay.

### BRIEF DESCRIPTION OF DRAWINGS

FIG. 1 illustrates the detection of Tip47 associated with lipid droplets.

FIG. 2 illustrates the detection of the reduction in the number of lipid droplets by detecting fluorescence of a PAT family protein.

FIG. 3 illustrates amino acid sequence of human perilipin.

FIG. 4 illustrates amino acid sequence of human ADFP.

FIG. 5 illustrates amino acid sequence of human Tip47.

FIG. 6 illustrates amino acid sequence of human S3-12.

FIG. 7 illustrates amino acid sequence of human PAT1.

FIG. 8 illustrates down-regulation of both ADFP and Tip47 with specific siRNAs drastically affects lipid droplet morphology in HEPG2 cells. Cells were transfected with either negative siRNA or with both siRNAs ADFP and Tip47 one day after plating. Cells were incubated with oleic acid overnight, with 400 mM FFA three days post-plating, and the next day cells were fixed with 3% paraformaldehyde and stained for lipid droplet and nuclei with Bodipy and Hoechst.

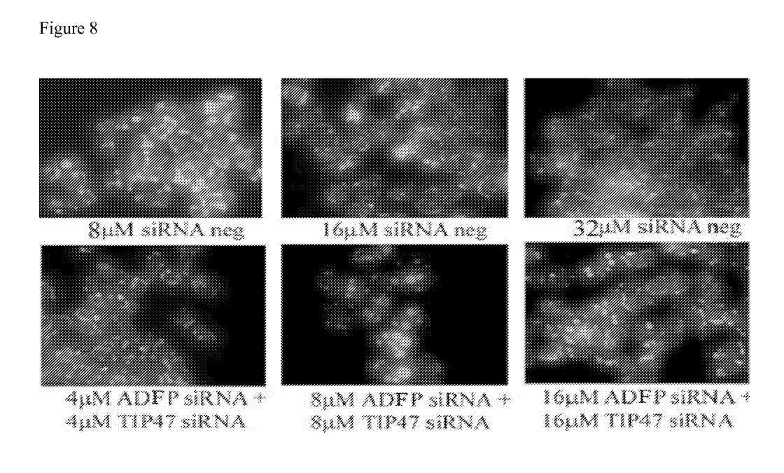

### DETAILED DESCRIPTION OF THE INVENTION

### I. Definitions

As used herein the specification, “a”, “an”, or “the” may mean one or more. As used herein in the claim(s), when used in conjunction with the word “comprising”, the words “a”, “an”, or “the” may mean one or more than one. As used herein “another” may mean at least a second or more.

As used herein, “about” refers to numeric values whether or not explicitly indicated. The term “about” generally refers to a range of numbers (e.g., +/−5-10% of the recited value) that one would consider equivalent to the recited value (i.e., having the same function or result). In many instances, the term “about” may include numbers that are rounded to the nearest significant figure.

### II. The Present Invention

A. Methods of Quantifying Lipid Droplets in a Cell

In certain embodiments, the invention relates to a method of quantifying lipid droplets in a cell. The quantification of lipid droplets can be determined by alterations in lipid droplet intensity, number, size, morphology, quantity, quality, or other phenotypic properties of a lipid droplet. A cell includes a mammalian and non-mammalian cell. A mammalian cell includes, for example, a cell derived from human, nonhuman primate, rat, mouse, guinea pig, rabbit, swine, sheep, bovine, Chinese hamster ovary (CHO), COS cells, etc.

In further embodiments, the invention is drawn to a method of measuring the expression level of a PAT family protein and comparing said expression level with a known standard, thereby quantifying lipid droplets in a cell. Detection of a PAT family protein can be either by direct or indirect means.

As will be appreciated by one of ordinary skill in the art, any means for specifically identifying and determining quantitatively the amount or concentration of a PAT family protein is considered. A particular method for detecting a PAT family protein in a cell is by means of using antibodies, which is an indirect method. An antibody to a particular PAT family protein, a primary antibody, is prepared for use by the skilled artisan. Polyclonal or monoclonal antibodies that are specific for a PAT family protein is prepared for use by the skilled artisan. Detection of an antibody that binds to a PAT family protein is determined by, for example, a labeled secondary antibody. In one embodiment, the secondary antibody is fluorescently labeled. In another embodiment, the secondary antibody is enzymatically labeled.

A variety of assays are available for detecting polypeptides with a labeled antibody. For example, in a two-step assay, a cell comprising a PAT family protein is incubated with an unlabeled primary antibody that binds to said protein, thereby forming an immune complex. The bound unlabeled primary antibody is then incubated with a secondary labeled antibody that is specific for the unlabeled primary antibody and binds thereto. Unbound molecules are washed away (i.e., removed) and the presence of the secondary labeled antibody is detected. Alternatively, in a one-step assay, a PAT family protein is immobilized and incubated with a labeled antibody. The labeled antibody binds to the immobilized PAT family protein. Unbound molecules are washed away (i.e., removed) and the presence of the label is detected. The choice of label on the antibodies may be a fluorescent moiety, an enzyme, a chromophoric moiety, a radioactive atom, a biotin tag, or a calorimetric tag. Some examples of a fluorescent moiety include rhodamine, fluorescein, TEXAS RED, etc. Some examples of enzymes include, horseradish peroxidase, glucose oxidase, glucose-6-phosphate dehydrogenase, alkaline phosphatase, beta-galactosidase, urease, luciferase, etc. Some examples of radioactive atoms are ³²P, ¹²⁵I, ³H, etc. An Enzyme Linked ImmunoSorbent Assay (ELISA) allows detection of an enzyme-complex with a substrate that produces a detectable product. The method of detection will depend on the particular type of labeled antibody used. Detection may include any method known in the art that has, or may have been adapted, to detect a particular labeled antibody, including, for example, fluorescence, luminescence, ELISA, radiography, etc.

In addition to the indirect method described above, a PAT family protein may be detected by direct methods. Direct methods include, for example, the use of a PAT family protein fusion protein. A used in this method, a fusion protein would comprise a PAT family protein, or a functional portion thereof, fused to a detectable molecule (e.g., green fluorescent protein). As described herein, a functional portion comprises a fragment of a full-length PAT family protein that associates with a lipid droplet in a cell and may be detected according to methods of the invention. Once clonal cells comprising the fusion protein have been established, as described in Example 2, direct measurement of the PAT family protein fusion protein is carried out (i.e., there is no use of indirect measures).

Quantification of lipid droplets comprises comparing the expression level of a PAT family protein. In one embodiment, the expression level of a PAT family protein consists of comparing a cell to a known standard. A change in the level the PAT family protein, as compared to the known standard, is indicative of a change in the quantity of lipid droplets. A known standard can, for example, be established by quantifying varying known amounts of lipid droplets and assigning a value to said known amount (e.g., generating a standard curve using quantifiable fluorescence or chemiluminescence intensity).

B. PAT Family Protein

The mammalian PAT family of proteins contains at least five members (perilipin [SEQ ID NO: 1], ADFP [SEQ ID NO: 2], Tip47 [SEQ ID NO: 3], S3-12 [SEQ ID NO: 4], and PAT1 [SEQ ID NO: 5]). PAT family proteins are defined by primary sequence homology and are well conserved within a species and across species. A PAT family protein refers to any PAT family protein within a species or any PAT family protein in another species so long as said protein associates with a lipid droplet in a cell and may be detected according to methods of the invention. A PAT family protein can mean the full-length sequence, a conserved domain, a fusion protein, or any functional portion thereof. Regardless of the PAT family protein used, the use of any such protein (e.g., PAT family protein fusion protein, PAT family protein functional portion thereof, etc.) refers to a peptide or protein comprising a minimum sequence whereby the peptide or protein associates with a lipid droplet in a cell and may be detected according to methods of the invention. In certain embodiments, a PAT family protein is full-length ADFP or is full-length ADFP fused with eGFP. In other certain embodiments, a PAT family protein is full-length Tip47 or is full-length Tip47 fused with eGFP.

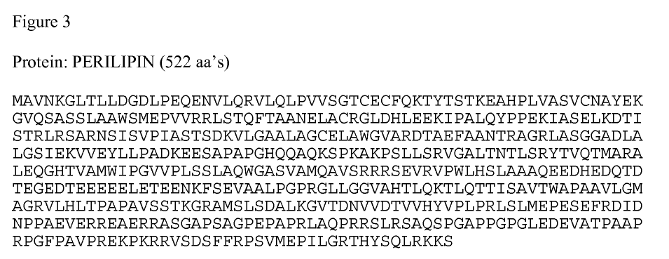

In certain embodiments, a functional portion of perilipin comprises a fragment of the full-length sequence that associates with a lipid droplet in a cell and may be detected according to methods of the invention. In specific embodiments, a functional portion of perilipin comprises at least about 5-10, about 11-15, about 16-20, about 21-25, about 26-30, about 31-35, about 36-40, about 41-45, about 46-50, about 51-55, about 56-60, about 61-65, about 66-70, about 71-75, about 76-80, about 81-85, about 86-90, about 91-95, about 96-100, about 101-105, about 106-110, about 111-115, about 116-120, about 121-125, about 126-130, about 131-135, about 136-140, about 141-145, about 146-150, about 151-155, about 156-160, about 161-165, about 166-170, about 171-175, about 176-180, about 181-185, about 186-190, about 191-195, about 196-200, about 201-205, about 206-210, about 211-215, about 216-220, about 221-225, about 226-230, about 231-235, about 236-240, about 241-245, about 246-250, about 251-255, about 256-260, about 261-265, about 266-270, about 271-275, about 276-280, about 281-285, about 286-290, about 291-295, about 296-300, about 301-305, about 306-310, about 311-315, about 316-320, about 321-325, about 326-330, about 331-335, about 336-340, about 341-345, about 346-350, about 351-355, about 356-360, about 361-365, about 366-370, about 371-375, about 376-380, about 381-385, about 386-390, about 391-395, about 396-400, about 401-405, about 406-410, about 411-415, about 416-420, about 421-425, about 426-430, about 431-435, about 436-440, about 441-445, about 446-450, about 451-455, about 456-460, about 461-465, about 466-470, about 471-475, about 476-480, about 481-485, about 486-490, about 491-495, about 496-500, about 501-505, about 506-510, about 511-515, or about 516-521 consecutive amino acids of SEQ ID NO: 1 (FIG. 3). In further embodiments, a functional portion of perlipin comprises the perilipin region/domain of SEQ ID NO: 1 (FIG. 3). In specific embodiments, the perilipin region/domain of perilipin comprises amino acid residues 7-398 of SEQ ID NO: 1 (FIG. 3).

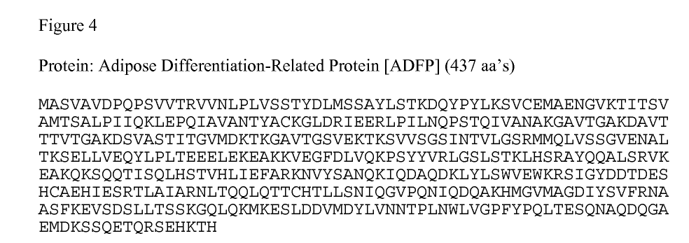

In certain embodiments, a functional portion of ADFP comprises a fragment of the full-length sequence that associates with a lipid droplet in a cell and may be detected according to methods of the invention. In specific embodiments, a functional portion a functional portion of ADFP comprises at least about 5-10, 11-15, about 16-20, about 21-25, about 26-30, about 31-35, about 36-40, about 41-45, about 46-50, about 51-55, about 56-60, about 61-65, about 66-70, about 71-75, about 76-80, about 81-85, about 86-90, about 91-95, about 96-100, about 101-105, about 106-110, about 111-115, about 116-120, about 121-125, about 126-130, about 131-135, about 136-140, about 141-145, about 146-150, about 151-155, about 156-160, about 161-165, about 166-170, about 171-175, about 176-180, about 181-185, about 186-190, about 191-195, about 196-200, about 201-205, about 206-210, about 211-215, about 216-220, about 221-225, about 226-230, about 231-235, about 236-240, about 241-245, about 246-250, about 251-255, about 256-260, about 261-265, about 266-270, about 271-275, about 276-280, about 281-285, about 286-290, about 291-295, about 296-300, about 301-305, about 306-310, about 311-315, about 316-320, about 321-325, about 326-330, about 331-335, about 336-340, about 341-345, about 346-350, about 351-355, about 356-360, about 361-365, about 366-370, about 371-375, about 376-380, about 381-385, about 386-390, about 391-395, about 396-400, about 401-405, about 406-410, about 411-415, about 416-420, about 421-425, about 426-430, or about 431-436 consecutive amino acids of SEQ ID NO: 2 (FIG. 4). In further embodiments, a functional portion of ADFP comprises the perilipin region/domain of SEQ ID NO: 2 (FIG. 4). In specific embodiments, the perilipin region/domain of ADFP comprises amino acid residues 2-396 of SEQ ID NO: 2 (FIG. 4).

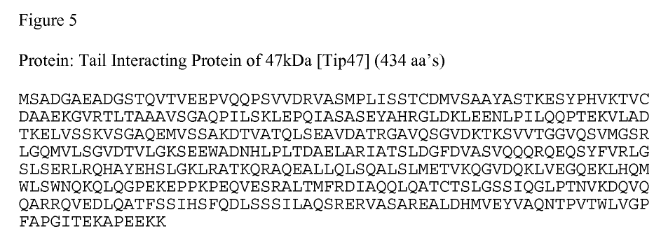

In certain embodiments, a functional portion of Tip47 comprises comprises a fragment of the full-length sequence that associates with a lipid droplet in a cell and may be detected according to methods of the invention. In specific embodiments, a functional portion of Tip47 comprises at least about 5-10, about 11-15, about 16-20, about 21-25, about 26-30, about 31-35, about 36-40, about 41-45, about 46-50, about 51-55, about 56-60, about 61-65, about 66-70, about 71-75, about 76-80, about 81-85, about 86-90, about 91-95, about 96-100, about 101-105, about 106-110, about 111-115, about 116-120, about 121-125, about 126-130, about 131-135, about 136-140, about 141-145, about 146-150, about 151-155, about 156-160, about 161-165, about 166-170, about 171-175, about 176-180, about 181-185, about 186-190, about 191-195, about 196-200, about 201-205, about 206-210, about 211-215, about 216-220, about 221-225, about 226-230, about 231-235, about 236-240, about 241-245, about 246-250, about 251-255, about 256-260, about 261-265, about 266-270, about 271-275, about 276-280, about 281-285, about 286-290, about 291-295, about 296-300, about 301-305, about 306-310, about 311-315, about 316-320, about 321-325, about 326-330, about 331-335, about 336-340, about 341-345, about 346-350, about 351-355, about 356-360, about 361-365, about 366-370, about 371-375, about 376-380, about 381-385, about 386-390, about 391-395, about 396-400, about 401-405, about 406-410, about 411-415, about 416-420, about 421-425, about 426-430, or about 431-433 consecutive amino acids of SEQ ID NO: 3 (FIG. 5). In further embodiments, a functional portion of Tip47 comprises the perilipin region/domain of SEQ ID NO: 3 (FIG. 5). In specific embodiments, the perilipin region/domain of Tip47 comprises amino acid residues 14-412 of SEQ ID NO: 3(FIG. 5).

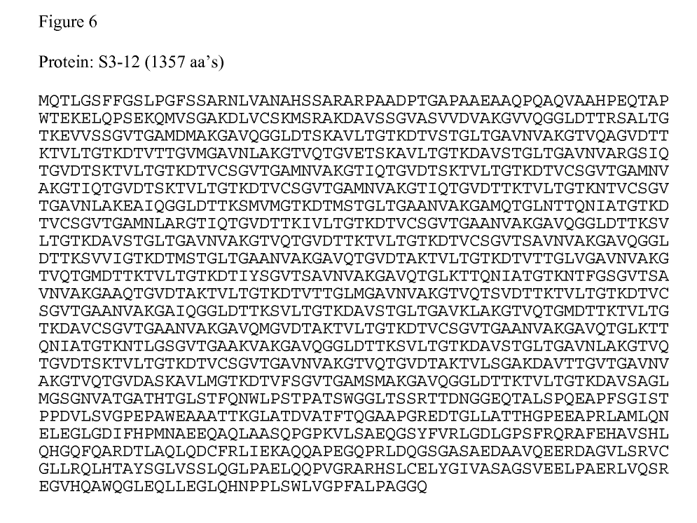

In certain embodiments, a functional portion of S3-12 comprises a fragment of the full-length sequence that associates with a lipid droplet in a cell and may be detected according to methods of the invention. In specific embodiments, a functional portion of S3-12 comprises at least about 5-10, about 11-15, about 16-20, about 21-25, about 26-30, about 31-35, about 36-40, about 41-45, about 46-50, about 51-55, about 56-60, about 61-65, about 66-70, about 71-75, about 76-80, about 81-85, about 86-90, about 91-95, about 96-100, about 101-105, about 106-110, about 111-115, about 116-120, about 121-125, about 126-130, about 131-135, about 136-140, about 141-145, about 146-150, about 151-155, about 156-160, about 161-165, about 166-170, about 171-175, about 176-180, about 181-185, about 186-190, about 191-195, about 196-200, about 201-205, about 206-210, about 211-215, about 216-220, about 221-225, about 226-230, about 231-235, about 236-240, about 241-245, about 246-250, about 251-255, about 256-260, about 261-265, about 266-270, about 271-275, about 276-280, about 281-285, about 286-290, about 291-295, about 296-300, about 301-305, about 306-310, about 311-315, about 316-320, about 321-325, about 326-330, about 331-335, about 336-340, about 341-345, about 346-350, about 351-355, about 356-360, about 361-365, about 366-370, about 371-375, about 376-380, about 381-385, about 386-390, about 391-395, about 396-400, about 401-405, about 406-410, about 411-415, about 416-420, about 421-425, about 426-430, about 431-435, about 436-440, about 441-445, about 446-450, about 451-455, about 456-460, about 461-465, about 466-470, about 471-475, about 476-480, about 481-485, about 486-490, about 491-495, about 496-500, about 501-505, about 506-510, about 511-515, about 516-520, about 521-525, about 526-530, about 531-535, about 536-540, about 541-545, about 546-550, about 551-555, about 556-560, about 561-565, about 566-570, about 571-575, about 576-580, about 581-585, about 586-590, about 591-595, about 596-600, about 601-605, about 606-610, about 611-615, about 616-620, about 621-625, about 626-630, about 631-635, about 636-640, about 641-645, about 646-650, about 651-655, about 656-660, about 661-665, about 666-670, about 671-675, about 676-680, about 681-685, about 686-690, about 691-695, about 696-700, about 701-705, about 706-710, about 711-715, about 716-720, about 721-725, about 726-730, about 731-735, about 736-740, about 741-745, about 746-750, about 751-755, about 756-760, about 761-765, about 766-770, about 771-775, about 776-780, about 781-785, about 786-790, about 791-795, about 796-800, about 801-805, about 806-810, about 811-815, about 816-820, about 821-825, about 826-830, about 831-835, about 836-840, about 841-845, about 846-850, about 851-855, about 856-860, about 861-865, about 866-870, about 871-875, about 876-880, about 881-885, about 886-890, about 891-895, about 896-900, about 901-905, about 906-910, about 911-915, about 916-920, about 921-925, about 926-930, about 931-935, about 936-940, about 941-945, about 946-950, about 951-955, about 956-960, about 961-965, about 966-970, about 971-975, about 976-980, about 981-985, about 986-990, about 991-995, about 996-1000, about 1001-1005, about 1006-1010, about 1011-1015, about 1016-1020, about 1021-1025, about 1026-1030, about 1031-1035, about 1036-1040, about 1041-1045, about 1046-1050, about 1051-1055, about 1056-1060, about 1061-1065, about 1066-1070, about 1071-1075, about 1076-1080, about 1081-1085, about 1086-1090, about 1091-1095, about 1096-1100, about 1101-1105, about 1106-1110, about 1111-1115, about 1116-1120, about 1121-1125, about 1126-1130, about 1131-1135, about 1136-1140, about 1141-1145, about 1146-1150, about 1151-1155, about 1156-1160, about 1161-1165, about 1166-1170, about 1171-1175, about 1176-1180, about 1181-1185, about 1186-1190, about 1191-1195, about 1196-1200, about 1201-1205, about 1206-1210, about 1211-1215, about 1216-1220, about 1221-1225, about 1226-1230, about 1231-1235, about 1236-1240, about 1241-1245, about 1246-1250, about 1251-1255, about 1256-1260, about 1261-1265, about 1266-1270, about 1271-1275, about 1276-1280, about 1281-1285, about 1286-1290, about 1291-1295, about 1296-1300, about 1301-1305, about 1306-1310, about 1311-1315, about 1316-1320, about 1321-1325, about 1326-1330, about 1331-1335, about 1336-1340, about 1341-1345, about 1346-1350, or about 1351-1356 consecutive amino acids of SEQ ID NO: 4 (FIG. 6). In further embodiments, a functional portion of S3-12 comprises the perilipin region/domain of SEQ ID NO: 4 (FIG. 6). In specific embodiments, the perilipin region/domain of S3-12 comprises amino acid residues 1096-1323 of SEQ ID NO: 4 (FIG. 6).

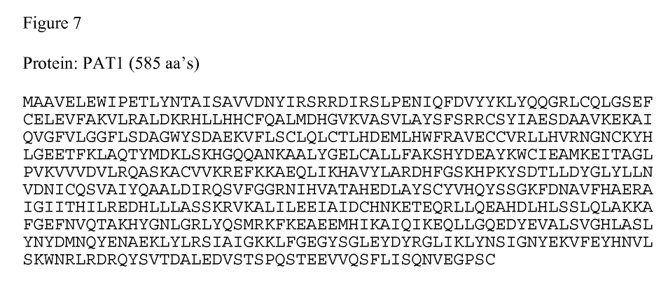

In certain embodiments, a functional portion of PAT 1 comprises a fragment of the full-length sequence that associates with a lipid droplet in a cell and may be detected according to methods of the invention. In specific embodiments, a functional portion of PAT1 comprises at least about 5-10, about 11-15, about 16-20, about 21-25, about 26-30, about 31-35, about 36-40, about 41-45, about 46-50, about 51-55, about 56-60, about 61-65, about 66-70, about 71-75, about 76-80, about 81-85, about 86-90, about 91-95, about 96-100, about 101-105, about 106-110, about 111-115, about 116-120, about 121-125, about 126-130, about 131-135, about 136-140, about 141-145, about 146-150, about 151-155, about 156-160, about 161-165, about 166-170, about 171-175, about 176-180, about 181-185, about 186-190, about 191-195, about 196-200, about 201-205, about 206-210, about 211-215, about 216-220, about 221-225, about 226-230, about 231-235, about 236-240, about 241-245, about 246-250, about 251-255, about 256-260, about 261-265, about 266-270, about 271-275, about 276-280, about 281-285, about 286-290, about 291-295, about 296-300, about 301-305, about 306-310, about 311-315, about 316-320, about 321-325, about 326-330, about 331-335, about 336-340, about 341-345, about 346-350, about 351-355, about 356-360, about 361-365, about 366-370, about 371-375, about 376-380, about 381-385, about 386-390, about 391-395, about 396-400, about 401-405, about 406-410, about 411-415, about 416-420, about 421-425, about 426-430, about 431-435, about 436-440, about 441-445, about 446-450, about 451-455, about 456-460, about 461-465, about 466-470, about 471-475, about 476-480, about 481-485, about 486-490, about 491-495, about 496-500, about 501-505, about 506-510, about 511-515, about 516-520, about 521-525, about 526-530, about 531-535, about 536-540, about 541-545, about 546-550, about 551-555, about 556-560, about 561-565, about 566-570, about 571-575, about 576-580, or about 581-584 consecutive amino acids of SEQ ID NO: 5 (FIG. 7). In other certain embodiments, a functional portion of PAT1 comprises the perilipin region/domain of SEQ ID NO: 5 (FIG. 7).

In certain embodiments, conservative amino substitutions (e.g., a hydrophobic amino acid substitution for a hydrophobic amino acid in the wild-type sequence), comprising natural and synthetic amino acids, can be made to a PAT family peptide or protein, as set forth throughout the specification, of the invention so long as the peptide or protein associates with a lipid droplet and may be detected according to methods of the invention.

In further certain embodiments, a PAT family peptide or protein, as set forth throughout the specification, can be derived from various species, including prokaryotes and eukaryotes, so long as the peptide or protein associates with a lipid droplet and may be detected according to methods of the invention.

C. Screening Agents for the Ability to Regulate Lipid Metabolism

In certain embodiments, the invention relates to compositions and methods for screening an agent for the ability to modulate expression of genes and to regulate lipid metabolism in a cell. In specific embodiments, the method comprises contacting a first cell with an agent and measuring the expression level of a PAT family protein in said first cell, and comparing said expression level of the first cell with the expression level of a PAT family protein in a second cell not contacted with the agent, wherein a difference between PAT family protein expression levels measured in said first and second cells indicates that the agent modulates expression of the PAT family protein and regulates metabolism including lipid metabolism and insulin metabolism. An agent can be any molecular entity (e.g., a small molecule, nucleic acid, such as siRNA or shRNA expression cassette, protein, peptide, antibody, or other biomolecule that is naturally made, synthetically made, or semi-synthetically made) used alone or in combination with other molecular entities or treatments that can alleviate, reduce, ameliorate, prevent, or maintain in a state of remission clinical symptoms or diagnostic markers associated with undesired and/or uncontrolled diseases or disorders related to lipid metabolism.

Screening an agent for the ability to regulate lipid metabolism comprises comparing the expression level of a PAT family protein. In one embodiment, the expression level of a PAT family protein consists of comparing a first cell contacted with an agent against a second cell not contacted with the agent. A difference in the expression level of a PAT family protein in the first cell contacted with an agent compared to a second cell not contacted with the agent indicates that said agent regulates lipid metabolism. Under this paradigm, everything is held constant except for the first cell being contacted with an agent and the second cell not being contacted with the agent. For example, the first and second cells are from the same source, are of the same age, and are housed under identical conditions throughout.

In certain embodiments, lipid droplet screening is carried out as described throughout the specification, but is alternatively accomplished by staining of lipid droplets with BODIPY lipid probes (Invitrogen Molecular Probes, Eugene, Oreg.) or equivalents that also stain lipid molecules. In specific embodiments, BODIPY lipid probes stain for fatty acids, glycerophospholipids with labeled acyl chains, glycerophospholipids with labeled head groups, cholesteryl esters, or neutral lipids. In specific embodiments, BODIPY lipid probes stain for neutral lipids. In further specific embodiments, BODIPY lipid probes are BODIPY493/503, BODIPY 505/515, or BODIPY 558/568.

Regulating lipid metabolism refers to altering lipid metabolism. For example, altered lipid metabolism can be an increase or a decrease in the rate of lipolysis or an increase or a decrease in the rate of lipid deposition. Additionally, altering lipid metabolism includes an interaction with a signaling cascade molecule or pathway. An interaction with a signaling cascade molecule or pathway can, for example, result in an increase or a decrease in the rate of lipolysis or an increase or decrease in the rate of lipid deposition.

D. High Throughput Screening

Methods described herein are adaptable to high throughput screening methods. A high throughput assay design allows screening or analyzing of multiple samples simultaneously. A high throughput assay has the capacity for being carried out and/or manipulated by robotics, for at least one step of the assay. A desired feature of high throughput assays is, for example, a screening method optimized to screen large numbers of samples, reduced assay time, reduced reagent amount, reduced sample amount, or minimization of the number of manipulations in order to achieve the analysis desired. Assay formats include, for example, 96-well or 384-well plates, levitating droplets, and chip technology, such as, microchannel chips used for liquid handling experiments. It is well known by one of ordinary skill in the art that as technology in the field of high throughput screening advances (e.g., miniaturization of plastic molds, liquid handling devices are improved, etc.) or as improved assay methods or devices are designed, that greater numbers of samples can be analyzed, assay time can be reduced, and/or assay accuracy can be improved. The method of high throughput screening can be adapted as technology advances and is, therefore, not to be construed to limit the invention disclosed herein.

E. Treating a Disease or Disorder Related to Lipid Metabolism

In certain embodiments, the invention relates to methods of treating a disease or disorder related to lipid metabolism comprising administering to a patient in need thereof one or more of the agents identified by the methods described herein. In a further embodiment, the invention is drawn to the method wherein a disease or disorder related to lipid metabolism is selected from the group consisting of obesity, diabetes [non-insulin and insulin dependent], hypertension, coronary artery disease, hyperlipidemia (e.g., LDL, TAGs), hypolipidemia (e.g., HDL), lipid metabolism disorders, lipid deposition disorders, lipodystrophies. Treating a disease or disorder related to lipid metabolism includes alleviating, reducing, ameliorating, preventing, or maintaining in a state of remission clinical symptoms or diagnostic markers associated with undesired and/or uncontrolled diseases or disorders related to lipid metabolism. Diseases or disorders related to lipid metabolism refer to pathological conditions resulting from altered lipid metabolism (e.g., increases in lipid deposition), which can, for example, be a result of biochemical alterations, such as, the inability to secrete or respond to insulin, or a result of lifestyle choices, such as excess intake of fats. The term includes, for example, the following diseases or disorders: obesity, diabetes [non-insulin and insulin dependent], hypertension, coronary artery disease, hyperlipidemia (e.g., LDL, TAGs), hypolipidemia (e.g., HDL), lipid metabolism disorders, lipid deposition disorders, lipodystrophies.

An agent or agents used to treat a disease or disorder related to lipid metabolism can be used alone, in combination with one or more agents, or in combination with a therapy used to treat a disease or disorder related to lipid metabolism.

In one embodiment, treating obesity includes using an agent alone, in combination with one or more agents, or in combination with dietary modification or restriction, physical activity level modification, behavioral therapy, a surgical procedure (e.g., gastric by-pass, liposuction, etc.), a dietary supplement, gene therapy (e.g., gene therapy directed to defects in lipid metabolism enzymes, etc.), or a drug (e.g., orlistat (Xenical), sibutramine (Meridia), phendimetrazine, phentermine, diethylpropion, etc.).

In another embodiment, treating diabetes includes using an agent alone, in combination with one or more agents, or in combination with dietary modification or restriction, physical activity level modification, behavioral therapy, a dietary supplement, gene therapy (e.g., gene therapy directed to insulin secreting cells, gene therapy directed to insulin receptors, etc.), or a drug (e.g., Apidra, Exubera Combination Pack 12, Exubera Combination Pack 15, Exubera Kit, Humalog Mix 50-50, Humalog Mix 75-25, Humalog Pen, Humalog, Humulin 50/50, Humulin 70/30 Pen, Humulin 70/30, Humulin N Pen, Humulin N, Humulin R, Insulin Asp Prt-Insulin Aspart, Insulin Aspart, Insulin Detemir, Insulin Glargine, Insulin Glulisine, Insulin Inhalation Combo Pack, Insulin Inhalation Kit, Insulin Lisp & Lisp Prot (Hum), Insulin Lispro (Human), Insulin NPH Human Recombinant, Insulin NPH-Regular Human Recombinant, Insulin Regular Human, Insulin Regular Human, Lantus, Levemir Flexpen, Levemir, Novolin 70/30 InnoLet, Novolin 70/30 PenFill, Novolin 70/30, Novolin N InnoLet, Novolin N PenFill, Novolin N, Novolin R, Novolin R InnoLet, Novolin R PenFill, Novolog Flexpen, Novolog Mix 70-30 FlexPen, Novolog Mix 70-30, Novolog, Pramlintide, SYMLIN, Acarbose, Acetohexamide, actoplus met, Actos, Amaryl, AVANDAMET, Avandaryl, Avandia, Chlorpropamide, Diabeta, Diabinese, DUETACT, FORTAMET, Glimepiride, Glipizide, Glipizide-Metformin, Glucophage, Glucophage XR, Glucotrol, Glucotrol XL, Glucovance, Glumetza, Glyburide Micronized, Glyburide, Glyburide-Metformin, Glycron, Glynase, Glyset, JANUVIA, Metaglip, Metformin, Micronase, Miglitol, Nateglinide, Pioglitazone, Pioglitazone-Glimepiride, Pioglitazone-Metformin, Prandin, Precose, Repaglinide, RIOMET, Rosiglitazone, Rosiglitazone-Glimepiride, Rosiglitazone-Metformin, Sitagliptin, Starlix, Tolazamide, Tolbutamide, metformin, chlorpropamide, glipizide, glyburide, extenatide, pramlintidem, etc.).

In another embodiment, treating hypertension includes using an agent alone, in combination with one or more agents, or in combination with dietary modification or restriction, physical activity level modification, behavioral therapy, gene therapy (e.g., gene therapy directed to angiotension converting enzyme, gene therapy directed to vasodilating receptors, etc.), or a dietary supplement, or a drug (e.g., Accupril, Accuretic, Acebutolol, Aceon, Adalat CC, Afeditab CR, Aldactazide, Aldactone, Aldoclor, Aldomet, Aldoril D50, Aldoril-25, Altace, Amiloride, Amiloride-Hydrochlorothiazide, Amlodipine, Amlodipine-Atorvastatin, Amlodipine-Benazepril, Apresoline, Atacand HCT, Atacand, Atenolol, Atenolol-Chlorthalidone, Avalide, Avapro, Benazepril, Benazepril-Hydrochlorothiazide, Bendroflumethiazide, Benicar HCT, Benicar, Betaxolol, Bisoprolol Fumarate, Bisoprolol-Hydrochlorothiazide, Blocadren, Caduet, Calan, Calan SR, Candesartan, Candesartan-Hydrochlorothiazid, Capoten, Capozide, Captopril, Captopril-Hydrochlorothiazide, Cardene, Cardene, Cardene SR, Cardizem CD, Cardizem LA, Cardizem SR, Cardura, Cartia XT, Carvedilol, Catapres, Catapres-TTS-1 TD, Catapres-TTS-2 TD, Catapres-TTS-3 TD, Chlorothiazide, Chlorothiazide Sodium, Chlorthalidone, Clonidine, Clonidine TD, Clorpres, Coreg, Corgard, Corzide, Covera-HS, Cozaar, Demadex, Demadex, Dilacor XR, DILT-CD, Diltia XT, DILT-XR, Diovan HCT, Diovan, Diuril, Diuril, Doxazosin, Dyazide, DynaCirc CR, Enalapril Maleate, Enalapril Maleate-Felodipine, Enalaprilat, Enalapril-Hydrochlorothiazide, Enduron, Eplerenone, Eprosartan, Eprosartan-Hydrochlorothiazide, Felodipine, Fosinopril, Fosinopril-Hydrochlorothiazide, Furosemide, Guanabenz, Guanfacine, Hydrone, Hydralazine, Hydralazine, Hydralazine-Hydrochlorothiazid, Hydrochlorothiazide, Hydroflumethiazide, Hytrin, HYZAAR, Indapamide, Inderal LA, Inderal, Inderide, InnoPran XL, INSPRA, Inversine, Irbesartan, Irbesartan-Hydrochlorothiazide, Isoptin SR, Isradipine, Kerlone, Labetalol, Labetalol, Lasix, Levatol, Lexxel, Lisinopril, Lisinopril-Hydrochlorothiazide, Lopressor HCT, Lopressor, Losartan, Losartan-Hydrochlorothiazide, Lotensin HCT, Lotensin, Lotrel, Mavik, Maxzide, Maxzide-25 mg, Mecamylamine, Methyclothiazide, Methyldopa, Methyldopa-Chlorothiazide, Methyldopa-Hydrochlorothiazide, Methyldopate, Metolazone, Metoprolol Succinate, Metoprolol Tartrate, Metoprolol-Hydrochlorothiazide, Micardis HCT, Micardis, Microzide, Minipress, Minoxidil, Moduretic, Moexipril-Hydrochlorothiazide, Monopril HCT, Monopril, Nadolol, Nadolol-Bendroflumethiazide, Naturetin, Nicardipine, Nicardipine, Nifediac CC, Nifedical XL, Nifedipine, Nisoldipine, Nitroglycerin in D5W, Nitroglycerin, Nitropress, Normodyne, Normodyne, Norvasc, Olmesartan, Olmesartan-Hydrochlorothiazide, Penbutolol, Perindopril Erbumine, Pindolol, Plendil, Prazosin, Prinil, Prinzide, Procardia XL, Propranolol, Propranolol-Hydrochlorothiazid, Quinapril, Quinapril-Hydrochlorothiazide, Quinaretic, Ramipril, Reserpine, Saluron, Sectral, Spironolactone, Spironolacton-Hydrochlorothiaz, Sular, Tarka, Taztia XT, Telmisartan, Telmisartan-Hydrochlorothiazid, Tenex, Tenoretic 100, Tenoretic 50, Tenormin, Terazosin, Teveten HCT, Teveten, Thalitone, Tiazac, Timolide, Timolol Maleate, Timolol-Hydrochlorothiazide, Toprol XL, Torsemide, Torsemide, Trandate, Trandate, Trandolapril, Trandolapril-Verapamil, Triamterene-Hydrochlorothiazid, Uniretic, Unasc, Valsartan, Valsartan-Hydrochlorothiazide, Vaseretic, Vasotec, Verapamil, Verelan, Verelan PM, Zaroxolyn, Zebeta, Zestoretic, Zestril, Ziac, Betapace AF, Betapace, Bumetanide, Bumetanide, Bumex, Cardizem, Diltiazem HC1, Diltiazem HC1, Diltiazem in D5W, Dyrenium, Edecrin, Ethacrynate Sodium, Ethacrynic Acid, Sodium Edecrin, Sorine, Sotalol AF, Sotalol, Triamterene, Corlopam, Diazoxide, Fenoldopam, Hyperstat, HYZAAR, Losartan-Hydrochlorothiazide, Nitroglycerin, Nitropress, etc.).

In another embodiment, treating coronary artery disease includes using an agent alone, in combination with one or more agents, or in combination with dietary modification or restriction, physical activity level modification, behavioral therapy, a surgical procedure (e.g., coronary bypass, etc.), a dietary supplement, gene therapy (e.g., gene therapy directed to targets involved in arterial lipid deposition or foam cell formation, etc.), or a drug (e.g., Altoprev, Amlodipine-Atorvastatin, Atorvastatin, Caduet, Gemfibrozil, Lipitor, Lopid, Lovastatin, Mevacor, Pravachol, Pravastatin, Altoprev, Amlodipine, Fluvastatin, Lescol, Lescol XL, Lovastatin, Mevacor, Norvasc, Pravachol, Pravastatin, Simvastatin, Zocor, Endur-Acin, Inositol Niacinate, Niacels, Niacin, Niacinol, Niacor, NiaDelay, Niaspan, Slo-Niacin, etc.).

In another embodiment, treating hyperlipidemia includes using an agent alone, in combination with one or more agents, or in combination with dietary modification or restriction, physical activity level modification, behavioral therapy, a surgical procedure (e.g., angioplasty, etc.), a dietary supplement, gene therapy (e.g., directed to lipid or cholesterol metabolizing enzymes, etc.), or a drug (e.g Advicor, Altoprev, Amlodipine-Atorvastatin, Atorvastatin, Caduet, Cholestyramine Light, Cholestyramine-Aspartame, Cholestyramine-Sucrose, Colestid Flavored, Colestid, Colestipol, Endur-Acin, Gemfibrozil, Inositol Niacinate, Lipitor, Lopid, Lovastatin, Mevacor, Niacels, Niacin, Niacin-Lovastatin, Niacinol, Niacor, NiaDelay, Niaspan, Pravachol, Pravastatin, Prevalite, Questran Light, Questran, Simvastatin, Slo-Niacin, Vigorex, Yohimbine Combinations, Zocor Neo-Fradin, Neomycin, Tricor, Triglide, Vytorin 10/10, Vytorin 10/20, Vytorin 10/40, Vytorin 10/80, Zetia,Rosuvastatin, Policosanol, Lescol, Lescol XL, Lipex, Lofibra, Antara, Crestor, Ezetimibe, Ezetimibe-Simvastatin, Fenofibrate Micronized, Fenofibrate Nanocrystallized, Fenofibrate, Colesevelam, WelChol, etc.).

In another embodiment, treating hypolipidemia includes using an agent alone, in combination with one or more agents, or in combination with dietary modification or restriction, physical activity level modification, behavioral therapy, a dietary supplement, gene therapy, or a drug.

In another embodiment, treating lipid metabolism disorders includes using an agent alone, in combination with one or more agents, or in combination with dietary modification or restriction, physical activity level modification, behavioral therapy, a dietary supplement, gene therapy, or a drug.

In another embodiment, treating lipid deposition disorders includes using an agent alone, in combination with one or more agents, or in combination with dietary modification or restriction, physical activity level modification, behavioral therapy, a dietary supplement, gene therapy, or a drug.

F. Prognostic Test

In certain embodiments, the invention relates to the prognosis of a disease or disorder related to lipid metabolism. In further embodiments, the invention relates to the prognosis of the status (e.g., progression, maintenance, etc.) of a disease or disorder related to lipid metabolism in a subject already diagnosed as having said disease or disorder whereby lipid droplets in a cell are quantified. Comparative quantification of the sample is carried out using a cell (a second cell) obtained from a sample and a cell (a first cell) obtained from a like sample (e.g., a cell taken from a sample of the same organ as the second cell with the same spatial parameters) that was previously quantified and comparing the quantification of lipid droplets between the first and second cells. Alterations in quantified lipid droplets (e.g., morphology, size, etc.) form the basis of the prognosis. For example, if there is a difference in the quantity of lipid droplets (e.g., an increase in the quantity of lipid droplets) this could indicate that the disease or disorder related to lipid metabolism is progressing. In another example, if there is no difference in the quantity of lipid droplets this could indicate that the disease or disorder related to lipid metabolism is not progressing (i.e., said disease or disorder is maintaining). Under this paradigm, everything is held constant except the first cell and the second cell are not assayed at the same time. For example, the first and second cells are from the same sample (e.g., same liver lobe or lobule), and are housed under identical conditions throughout. Alternatively, the assay can be performed using procedures and controls readily known by one of ordinary skill in the art so as to eliminate variability between the first and second cells thereby allowing a comparison, and a conclusion based the comparison, of said cells.

In further embodiments, the invention relates to the prognosis of a potential therapy (e.g., an agent) for a disease or disorder related to lipid metabolism in a subject already diagnosed as having said disease or disorder whereby lipid droplets in a cell that are obtained from a sample are quantified. Comparative quantification of the cell is carried out using a first cell that is contacted with a potential therapy and a second cell that is not contacted with the potential therapy, followed by comparing the two cells. For example, if there is a difference in the quantity of lipid droplets (e.g., decreased quantity in the cell contacted with the potential therapy) this could indicate that the subject will respond to the potential therapy. In another example, if there is no difference in the quantity of lipid droplets this could indicate that the subject will not respond to the potential therapy. Under this paradigm, everything is held constant except the first cell is being contacted with a potential therapy and the second cell is not being contacted with the potential therapy. For example, the first and second cells are from the same sample (e.g., same liver lobe or lobule), and are housed under identical conditions throughout. Alternatively, the assay can be performed using procedures and controls readily known by one of ordinary skill in the art so as to eliminate variability between the first and second cells thereby allowing a comparison, and a conclusion based the comparison, of said cells.

As used herein, a “sample” refers typically to any type of material of biological origin including, but not limited to, cells, fluid, tissue, or organ isolated from a subject, including, for example, blood, plasma, serum, fecal matter, urine, semen, bone marrow, bile, spinal fluid, lymph fluid, samples of the skin, external secretions of the skin, respiratory, intestinal, and genitourinary tracts, tears, saliva, milk, blood cells, organs, or biopsies.

The foregoing examples illustrate the detection and/or use of particular PAT family proteins. However, the detection and/or use of a PAT family protein are not restricted to any particular PAT family protein. Additionally, the foregoing examples may use particular cells, which should not be construed as limiting the invention in any way. Furthermore, as previously discussed, an agent includes siRNA, to which particular examples may be drawn. However, methods comprising detecting a PAT family protein is not restricted to detecting a particular PAT family protein or using a particular agent or class of agents.

G. Identification of Genes involved in regulating lipid droplet morphology using siRNA and Lipid Droplet Detection

96 multiwell plates (Mattek) are coated with appropriate individual siRNA targeting one or more genes of interest, for example, siRNA directed to genes known or expected to have roles in a disease or disorder related to lipid metabolism in lipid metabolism, lipid droplet morphology, insulin sensitivity and signal transduction, and other physiological phenotypes of diabetes, many of which are important aspects of diabetes. Liver AML12 cells (mouse) and liver HEPG2 cells (human) (ATCC, Manassas, Va.) are plated, for example, at a concentration of 1×10⁴ for siRNA down-regulation and are transfected with HyPerFect (Qiagen). 48 hours later, half of the cells are treated with oleic acid (for example, 0-400 μM) overnight. siRNA are identified for the use as control (e.g., some of these siRNA can increase the amount of lipids, decrease the amount of lipids or change the lipid droplet morphology). siRNA identified for use as a control are used as an internal standard for each plate and are valuable tools to monitor and quantify the signal to noise ratio from one plate to another. For small drugs screens, each plate will include some wells treated with increasing amount of exogenous oleic acid and some of the wells will be in addition treated with Triacsin C (for example, 0-50 μM) (BIORAD, Hercules, Calif.) for 1 hour prior to FFA treatment. Triacsin C is a specific inhibitor of triglyceride accumulation. These controls are used on each plate as a means to help quantify the small drugs effects in decreasing the amount of lipids as well as helping to monitor the noise to signal ration among the different plates. Cells are fixed, for example, in 3% v/v paraformaldehyde for 30 min at room temperature. Neutral lipids are then stained with 4,4-difluoro-1,3,5,7,8-pentamethyl-4-bora-3a,4a-diaza-s-indacene (BODIPY 493/503) (Invitrogen Molecular Probes, Eugene, Oreg.) diluted in PBS (pH 7.4) and the nuclei are stained with Hoechst 33342 trihydrochloride tihydrate (Invitrogen Molecular Probes, Eugene, Oreg.) diluted in PBS (pH 7.4) for 20 min at room temperature. The concentrations for Bodipy and Hoechst staining are, for example, 0.15 mg/mL and 1.5 μg/mL for a 96 multiwell dish. For small drug screens we optimize the plating conditions so that the whole procedure from plating to readout is able to take place in less than 6 hours. AML-12 cells are plated with both the small drug and the NEFA (oleic acid 400 μM) in 96 multiwell plates, centrifuged at 800 g, and set in the incubator for 3 hours. Cells are then fixed and stained with Bodipy and Hoechst for an additional 1 hr. Procedures use, for example, an automated system from cellomix and algorithms are optimized to obtain the best quality of imaging as well as reproducibility. Small siRNA screens are performed initially using flexiplates from Qiagen.

### EXAMPLE 1

### Detecting PAT Family Proteins on Lipid Droplets

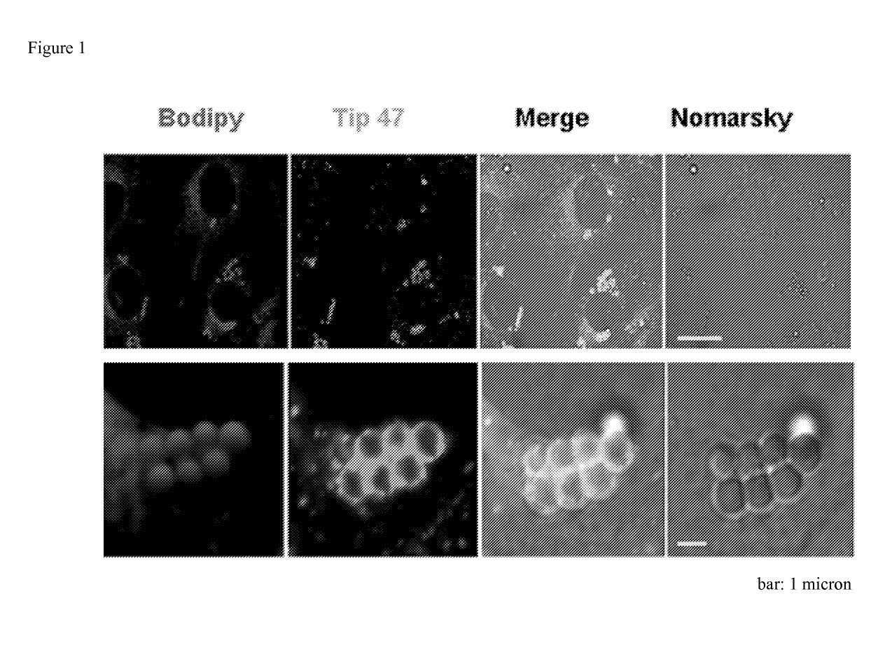

Anti-Tip47 antibodies were raised in rabbits against the COOH-terminal 200 amino acids of murine Tip47 and anti-ADFP were raised in rabbits against a peptide comprising the 26 amino-terminal amino acids of murine ADFP. Antibody specificity was confirmed with the use of two clonal CHO-K1 cell lines, stably transfected with the pEGFP-C2 vector containing coding murine ADFP and Tip47 sequences. Immunostaining revealed that the ADFP and Tip47 antibodies do not cross-react. Bodipy 558/568 and Bodipy 493/503 (Invitrogen) were used to visualize intracellular neutral lipid droplets. Alexa Fluor 488 and Alexa Fluor 598 species-specific secondary antibodies were purchased from Molecular Probes (Invitrogen). FIG. 1 illustrates the detection of Tip47 on associated with lipid droplets. Cells were stained with anti-Tip47 antibody and Alexa Fluor 488-conjugated secondary antibody.

### EXAMPLE 2

### PAT Family Protein Fusion Protein Transfection

Full-length Tip47 and ADFP cDNA constructs were fused in-frame with the 3′ end of eGFP in pEGFP-C2 (Clontech) (Miura et al., 2002, J. Biol. Chem. 277, 32253-32257). CHO cells were transfected with Lipofectamine, using either ADFP-GFP or Tip47-GFP fusion protein constructs. Clonal cell lines were established as described previously (Sztalryd et al., 2003, J. Cell Biol. 161, 1093-1103). Immediately following infection, CHO cells were placed under selection in 600 μg/ml G418, and cells after 8 days in selection medium were re-plated in 96-multiwell plates. Positive “green” clonal cells were identified by microscopy and further expanded. Analogous clonal cells derived from human or nonhuman primate is established in a like manner.

### EXAMPLE 3

### Detecting a PAT Family Proteins Following Exposure to an Agent

The siRNAs directed against mouse Tip47 cargo protein were designed by Qiagen. Cells were plated in antibiotic-free Dulbecco's modified Eagle's medium supplemented with 2% fetal calf serum at a concentration of 1×10⁴ cells per well in a 24-multiwell dish. During the next 2 days, cells were transfected with siRNAs (20 nM) using RNAiFect (Qiagen) according to the manufacturer's instructions. Among six siRNAs for our target gene, the sequences specific for Tip47 cargo protein sense (5′-AACAGCACAGAGAAUGAGGAG-3′) and sense (5′GAAUGAGACAUGUGUUUAA-3′) and were selected based upon their potency to inhibit target gene expression. Equal amounts of two positive siRNAs were used. An ineffective siRNA was used as a negative control, sense (5′-GCGUGUCCAAUCAGUCAU-3′). For lipogenesis experiments, wt and ADFP cells were transfected using Hyperfect (Qiagen) according to the manufacturer's instructions, cells were plated at a concentration of 1×10⁴ cells per well with Dulbecco's modified Eagle's medium supplemented with 10% fetal calf serum in a 24-multiwell dish, and transfection with siRNA was performed the next day. In all experiments, cells were used for Western blotting and immunocytochemistry on day 5 after plating unless otherwise stated, since preliminary experiments have shown that day 5 was optimal for Tip47 protein down-regulation (effect of Tip47 siRNA measured by Western blotting was decreased after day 7 post-plating).

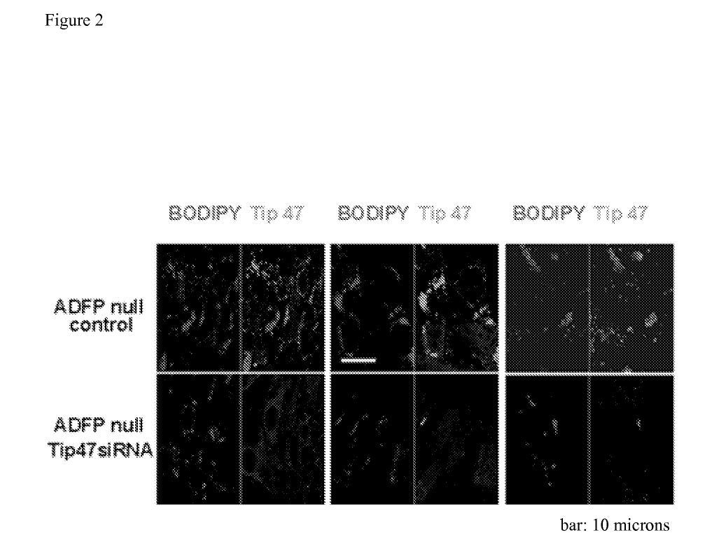

FIG. 2 illustrates the detection of the reduction in the number of lipid droplets by detecting fluorescence of a PAT family protein. ADFP null cells were transfected with negative control siRNA (top) or Tip47 siRNA (bottom) and 4 days later exposed overnight to 400 microM oleic acid. The following day, cells were fixed and co-stained with Bodipy 568, a neutral lipid stain (red), and Tip47 antibody (green). Fluorescent images were generated by LSM510 confocal laser microscopy.

### EXAMPLE 4

### Screening an Agent for the Ability to Regulate Lipid Metabolism

A potential agent, which may have the ability to regulate lipid metabolism is generated from a natural, synthetic, or semi-synthetic source. The agent is determined to demonstrate the ability to regulate lipid metabolism by measuring the amount of fluorescence of a PAT family protein in a cell under conditions where the agent is present compared to when the agent is not present, with all other parameters or conditions being held constant except for the presence or absence of the agent.

While the invention has been described with reference to certain particular embodiments thereof, those skilled in the art will appreciate that various modifications may be made without departing from the spirit and scope of the invention. Additionally, it is contemplated that any method or composition described herein can be implemented with respect to any other method or composition described herein. Furthermore, the scope of the appended claims is not to be limited to the specific embodiments described herein.

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in its entirety.

What is claimed:

1. A method of quantifying lipid droplets in a cell comprising the steps of,
(a) labeling lipid droplets in said cell with BODIPY;
(b) measuring the level of BODIPY staining in said cell; and
(b) comparing said level of BODIPY staining, with a known standard,
thereby quantifying lipid droplets in said cell.

2. A method of quantifying lipid droplets in a cell comprising the steps of,
(a) measuring the expression level of a PAT family protein; and
(b) comparing said expression level of a PAT family protein, with a known standard,
thereby quantifying lipid droplets in said cell.

3. The method of claim 2, wherein said PAT family protein is selected from the group consisting of:
a) perilipin;
b) adipose differentiation-related protein (ADFP);
c) tail interacting protein of 47 kDa (Tip47);
d) S3-12; and
e) PAT-1.

4. The method of claim 3, wherein said PAT family protein is adipose differentiation-related protein or tail interacting protein of 47 kDa.

5. The method of claim 2, wherein said PAT family protein is labeled.

6. The method of claim 5, wherein said label is a second polypeptide.

7. The method of claim 6, wherein said second polypeptide is selected from the group consisting of:
a) an enzyme; and
b) a fluorescent protein.

8. The method of claim 2, wherein said PAT family protein is detected using a technique selected from the group consisting of:
a) fluorescence;
b) luminescence;
c) ELISA;
d) radioimmunoassay.

9. A method of screening an agent for the ability to regulate lipid metabolism in a cell comprising the steps of,
(a) contacting a first cell with said agent;
(b) measuring the expression level of a PAT family protein in said first cell according to the method of claim 2; and
(c) comparing said expression level of a PAT family protein with the expression level of a PAT family protein in a second cell not contacted with said agent,
wherein a difference between PAT family protein expression levels measured in said frst and second cells indicates that said agent regulates lipid metabolism.

10. The method of claim 9, wherein said agent is a small inhibitory RNA.

11. The method of claim 9, wherein said cell is a human cell.

12. The method of claim 9, wherein said PAT family protein is selected from the group consisting of:
a) perilipin;
b) adipose differentiation-related protein (ADFP);
c) tail interacting protein of 47 kDa (Tip47);
d) S3-12; and
e) PAT-1.

13. The method of claim 12, wherein said PAT family protein is adipose differentiation-related protein or tail interacting protein of 47 kDa.

14. The method of claim 9, wherein said PAT family protein is labeled.

15. The method of claim 14, wherein said label is a second polypeptide.

16. The method of claim 15, wherein said second polypeptide is selected from the group consisting of:
a) an enzyme; and
b) a fluorescent protein.

17. The method of claim 9, wherein said PAT family protein is detected using a technique selected from the group consisting of:
a) fluorescence;
b) luminescence;
c) ELISA;
d) radioimmunoassay.

18. An agent identified by the method of claim 9 that is capable of regulating lipid metabolism in a cell.

19. A method of treating a disease or disorder related to lipid metabolism comprising administering to a patient in need thereof one or more agents identified according to method 9.

20. The method of claim 18, wherein said disease or disorder related to lipid metabolism is selected from the group consisting of obesity, diabetes [non-insulin and insulin dependent], hypertension, coronary artery disease, hyperlipidemia (e.g., LDL, TAGs), hypolipidemia (e.g., HDL), lipid metabolism disorders, lipid deposition disorders, lipodystrophies.


SEQUENCE LISTING (8 sequences)

SEQ ID NO: 1 - protein, 522 aa, Homo sapiens
MAVNKGLTLLDGDLPEQENVLQRVLQLPVVSGTCECFQKTYTSTKEAHPLVASVCNAYEKGVQSASSLAAWSMEPVVRRLSTQFTAANELACRGLDHLEEKIPALQYPPEKIASELKDTISTRLRSARNSISVPIASTSDKVLGAALAGCELAWGVARDTAEFAANTRAGRLASGGADLALGSIEKVVEYLLPADKEESAPAPGHQQAQKSPKAKPSLLSRVGALTNTLSRYTVQTMARALEQGHTVAMWIPGVVPLSSLAQWGASVAMQAVSRRRSEVRVPWLHSLAAAQEEDHEDQTDTEGEDTEEEEELETEENKFSEVAALPGPRGLLGGVAHTLQKTLQTTISAVTWAPAAVLGMAGRVLHLTPAPAVSSTKGRAMSLSDALKGVTDNVVDTVVHYVPLPRLSLMEPESEFRDIDNPPAEVERREAERRASGAPSAGPEPAPRLAQPRRSLRSAQSPGAPPGPGLEDEVATPAAPRPGFPAVPREKPKRRVSDSFFRPSVMEPILGRTHYSQLRKKS

SEQ ID NO: 2 - protein, 437 aa, Homo sapiens
MASVAVDPQPSVVTRVVNLPLVSSTYDLMSSAYLSTKDQYPYLKSVCEMAENGVKTITSVAMTSALPIIQKLEPQIAVANTYACKGLDRIEERLPILNQPSTQIVANAKGAVTGAKDAVTTTVTGAKDSVASTITGVMDKTKGAVTGSVEKTKSVVSGSINTVLGSRMMQLVSSGVENALTKSELLVEQYLPLTEEELEKEAKKVEGFDLVQKPSYYVRLGSLSTKLHSRAYQQALSRVKEAKQKSQQTISQLHSTVHLIEFARKNVYSANQKIQDAQDKLYLSWVEWKRSIGYDDTDESHCAEHIESRTLAIARNLTQQLQTTCHTLLSNIQGVPQNIQDQAKHMGVMAGDIYSVFRNAASFKEVSDSLLTSSKGQLQKMKESLDDVMDYLVNNTPLNWLVGPFYPQLTESQNAQDQGAEMDKSSQETQRSEHKTH

SEQ ID NO: 3 - protein, 434 aa, Homo sapiens
MSADGAEADGSTQVTVEEPVQQPSVVDRVASMPLISSTCDMVSAAYASTKESYPHVKTVCDAAEKGVRTLTAAAVSGAQPILSKLEPQIASASEYAHRGLDKLEENLPILQQPTEKVLADTKELVSSKVSGAQEMVSSAKDTVATQLSEAVDATRGAVQSGVDKTKSVVTGGVQSVMGSRLGQMVLSGVDTVLGKSEEWADNHLPLTDAELARIATSLDGFDVASVQQQRQEQSYFVRLGSLSERLRQHAYEHSLGKLRATKQRAQEALLQLSQALSLMETVKQGVDQKLVEGQEKLHQMWLSWNQKQLQGPEKEPPKPEQVESRALTMFRDIAQQLQATCTSLGSSIQGLPTNVKDQVQQARRQVEDLQATFSSIHSFQDLSSSILAQSRERVASAREALDHMVEYVAQNTPVTWLVGPFAPGITEKAPEEKK

SEQ ID NO: 4 - protein, 1357 aa, Homo sapiens
MQTLGSFFGSLPGFSSARNLVANAHSSARARPAADPTGAPAAEAAQPQAQVAAHPEQTAPWTEKELQPSEKQMVSGAKDLVCSKMSRAKDAVSSGVASVVDVAKGVVQGGLDTTRSALTGTKEVVSSGVTGAMDMAKGAVQGGLDTSKAVLTGTKDTVSTGLTGAVNVAKGTVQAGVDTTKTVLTGTKDTVTTGVMGAVNLAKGTVQTGVETSKAVLTGTKDAVSTGLTGAVNVARGSIQTGVDTSKTVLTGTKDTVCSGVTGAMNVAKGTIQTGVDTSKTVLTGTKDTVCSGVTGAMNVAKGTIQTGVDTSKTVLTGTKDTVCSGVTGAMNVAKGTIQTGVDTTKTVLTGTKNTVCSGVTGAVNLAKEAIQGGLDTTKSMVMGTKDTMSTGLTGAANVAKGAMQTGLNTTQNIATGTKDTVCSGVTGAMNLARGTIQTGVDTTKIVLTGTKDTVCSGVTGAANVAKGAVQGGLDTTKSVLTGTKDAVSTGLTGAVNVAKGTVQTGVDTTKTVLTGTKDTVCSGVTSAVNVAKGAVQGGLDTTKSVVIGTKDTMSTGLTGAANVAKGAVQTGVDTAKTVLTGTKDTVTTGLVGAVNVAKGTVQTGMDTTKTVLTGTKDTIYSGVTSAVNVAKGAVQTGLKTTQNIATGTKNTFGSGVTSAVNVAKGAAQTGVDTAKTVLTGTKDTVTTGLMGAVNVAKGTVQTSVDTTKTVLTGTKDTVCSGVTGAANVAKGAIQGGLDTTKSVLTGTKDAVSTGLTGAVKLAKGTVQTGMDTTKTVLTGTKDAVCSGVTGAANVAKGAVQMGVDTAKTVLTGTKDTVCSGVTGAANVAKGAVQTGLKTTQNIATGTKNTLGSGVTGAAKVAKGAVQGGLDTTKSVLTGTKDAVSTGLTGAVNLAKGTVQTGVDTSKTVLTGTKDTVCSGVTGAVNVAKGTVQTGVDTAKTVLSGAKDAVTTGVTGAVNVAKGTVQTGVDASKAVLMGTKDTVFSGVTGAMSMAKGAVQGGLDTTKTVLTGTKDAVSAGLMGSGNVATGATHTGLSTFQNWLPSTPATSWGGLTSSRTTDNGGEQTALSPQEAPFSGISTPPDVLSVGPEPAWEAAATTKGLATDVATFTQGAAPGREDTGLLATTHGPEEAPRLAMLQNELEGLGDIFHPMNAEEQAQLAASQPGPKVLSAEQGSYFVRLGDLGPSFRQRAFEHAVSHLQHGQFQARDTLAQLQDCFRLIEKAQQAPEGQPRLDQGSGASAEDAAVQEERDAGVLSRVCGLLRQLHTAYSGLVSSLQGLPAELQQPVGRARHSLCELYGIVASAGSVEELPAERLVQSREGVHQAWQGLEQLLEGLQHNPPLSWLVGPFALPAGGQ

SEQ ID NO: 5 - protein, 585 aa, Homo sapiens
MAAVELEWIPETLYNTAISAVVDNYIRSRRDIRSLPENIQFDVYYKLYQQGRLCQLGSEFCELEVFAKVLRALDKRHLLHHCFQALMDHGVKVASVLAYSFSRRCSYIAESDAAVKEKAIQVGFVLGGFLSDAGWYSDAEKVFLSCLQLCTLHDEMLHWFRAVECCVRLLHVRNGNCKYHLGEETFKLAQTYMDKLSKHGQQANKAALYGELCALLFAKSHYDEAYKWCIEAMKEITAGLPVKVVVDVLRQASKACVVKREFKKAEQLIKHAVYLARDHFGSKHPKYSDTLLDYGLYLLNVDNICQSVAIYQAALDIRQSVFGGRNIHVATAHEDLAYSCYVHQYSSGKFDNAVFHAERAIGIITHILREDHLLLASSKRVKALILEEIAIDCHNKETEQRLLQEAHDLHLSSLQLAKKAFGEFNVQTAKHYGNLGRLYQSMRKFKEAEEMHIKAIQIKEQLLGQEDYEVALSVGHLASLYNYDMNQYENAEKLYLRSIAIGKKLFGEGYSGLEYDYRGLIKLYNSIGNYEKVFEYHNVLSKWNRLRDRQYSVTDALEDVSTSPQSTEEVVQSFLISQNVEGPSC

SEQ ID NO: 6 - RNA, 21 nt, Artificial Sequence
  chemically synthesized siRNA
aacagcacagagaaugaggag

SEQ ID NO: 7 - RNA, 19 nt, Artificial Sequence
  chemically synthesized siRNA
gaaugagacauguguuuaa

SEQ ID NO: 8 - RNA, 18 nt, Artificial Sequence
  chemically synthesized siRNA
gcguguccaaucagucau